(19) United States
(12) Patent Application Publication
(10) Pub. No.: US 2023/0398160 A1
(43) Pub. Date: Dec. 14, 2023

(54) PROBIOTIC COMPOSITIONS AND METHODS AGAINST INTESTINAL BARRIER DYSFUNCTION AND HEAT STRESS

(71) Applicant: National University of Singapore, Singapore (SG)

(72) Inventors: Matthew Wook Chang, Singapore (SG); Koon Jiew Chua, Singapore (SG); Hua Ling, Singapore (SG); In Young Hwang, Singapore (SG)

(73) Assignee: National University of Singapore, Singapore (SG)

(21) Appl. No.: 18/251,199

(22) PCT Filed: Oct. 30, 2020
(86) PCT No.: PCT/SG2021/050662

§ 371 Date: Apr. 28, 2023

(30) Foreign Application Priority Data
Oct. 30, 2020 (SG) 10202010833R

(51) Int. Cl. A61K 35/747; A61K 35/744; A61K 35/741; A61P 1/00

(52) U.S. Cl. CPC A61K 35/747; A61K 35/744; A61K 35/741; A61P 1/00; A61K 2035/115

(57) ABSTRACT

The present invention relates to a composition comprising or consisting of two or more bacteria strains, formulated for the prophylaxis or treatment of intestinal barrier dysfunction and/or heat stress in a subject. More particularly, the composition comprises two or more of Lactobacillus reuteri MM2-3, Lactobacillus plantarum WCSF1, Streptococcus thermophilus B of R and Escherichia coli Nissle 1917. The present invention also includes uses of the invention to treat conditions such as inflammatory bowel disease (IBD) and heat stress.

### FIELD OF THE INVENTION

The present invention relates to a composition comprising or consisting of two or more bacteria strains, formulated for the prophylaxis or treatment of intestinal barrier dysfunction and/or heat stress in a subject. More particularly, the composition comprises two or more of Lactobacillus reuteri MM2-3, Lactobacillus plantarum WCSF1, Streptococcus thermophilus B of R and Escherichia coli Nissle 1917. The present invention also includes uses of the invention to treat conditions such as inflammatory bowel disease (IBD) and heat stress.

### BACKGROUND OF THE INVENTION

Probiotics are live microbes that confer beneficial effects to the host when administered in adequate amounts. Prolonged heat stress causes a symptom called “leaky gut” where an increased plethora of molecules from the gut lumen enter the bloodstream to cause various health problems. Heat stress also causes death in extreme cases.

A prolonged period of physical exertion subjects the whole body to several severe changes, caused by increased expression of acute-phase proteins and changes in hormonal releases, resulting in muscle damage, increased intestinal permeability, and even systemic inflammation [Clark, A. and Mach, N., J Int Soc Sports Nutr 13: 43 (2016)]. Specifically, individuals such as soldiers and athletes who are subjected to outdoor training especially under hot and humid conditions, often have heightened risk to heat-stress injuries. Heat stress can weaken the gut epithelium and allow entry of bacterial toxins in the gut into the otherwise sterile bloodstream, causing severe heat-stroke-related symptoms and even death [Moran, A. P., Prendergast, M. M. & Appelmelk, B. J. FEMS Immunol Med Microbiol 16: 105-115 (1996)].

Probiotics are known to be used for a variety of applications [Cheng, F. S., et al., World J Clin Cases 8: 1361-1384 (2020); Cinque, B. et al., PLoS One 11: e0163216, doi:10.1371/journal.pone.0163216 (2016); Kumar, M., et al., Am J Physiol Gastrointest Liver Physiol 312: G34-G45, doi:10.1152/ajpgi.00298.2016 (2017); Shing, C. M. et al., Eur J Appl Physiol 114: 93-103, doi:10.1007/s00421-013-2748-y (2014)].

There is a need for an improved probiotic that prevents, ameliorates or treats intestinal barrier dysfunction and/or performance-related issues from heat stress.

### SUMMARY OF THE INVENTION

This invention relates to a novel probiotics cocktail that improves the health status of individuals with intestinal barrier dysfunction and/or performance-related issues from heat stress. In this study, the inventors formulated a probiotics cocktail that improves intestinal barrier function and physical performance under heat stress. First, the inventors evaluated a panel of probiotic strains and verified the probiotic strains that strengthen the intestinal epithelial lining. Next, the inventors formulated a probiotics cocktail using the four best-performing strains, Lactobacillus reuteri MM2-3, Lactobacillus plantarum WCSF1, Streptococcus thermophilus B of R and Escherichia coli Nissle 1917. In an animal model, the 4-strain probiotic cocktail reduced intestinal permeability, promoted the expression of tight-junction proteins, and improved physical performance, indicating protective efficacy against heat stress. This protective efficacy was up to 5.5-fold higher than that of the individual strains. Our probiotics cocktail is applicable to addressing health issues that arise from intestinal barrier dysfunction and heat stress, and to promoting physical performance under heat stress.

In a first aspect, the present invention provides a composition comprising or consisting of two or more of Lactobacillus reuteri MM2-3, Lactobacillus plantarum WCSF1, Streptococcus thermophilus B of R and Escherichia coli Nissle 1917, formulated to prevent or treat intestinal barrier dysfunction and/or heat stress in a subject.

In some embodiments, the composition comprises or consists of Lactobacillus reuteri MM2-3, Lactobacillus plantarum WCSF1, Streptococcus thermophilus B of R and Escherichia coli Nissle 1917.

In some embodiments, the heat stress is at least in part due to physical exertion by the subject.

In some embodiments, the composition is formulated to prevent or treat intestinal barrier dysfunction and/or heat stress-induced gut permeability in said subject.

In some embodiments, the composition comprises a dose of about 10⁸ colony-forming units (CFU) of each strain.

In some embodiments, the subject is human.

In some embodiments, the composition is in the form of a gelatin capsule, pressed tablet, gel cap, liquid beverage or sachet.

In a second aspect, the present invention provides a method of treatment or prophylaxis comprising administering to a subject in need of such treatment or prophylaxis an efficacious amount of a composition of the first aspect.

In some embodiments, the subject has, or will get, intestinal barrier dysfunction and/or heat stress.

In some embodiments, the subject is administered a composition comprising at least 10⁸ to 10¹¹ CFU (colony-forming units) per day.

In some embodiments, the subject is administered a composition comprising at least 10⁸ CFU (colony-forming units) of each strain, preferably at least 5×10⁹ CFU (colony-forming units) of each strain.

In some embodiments, the composition lowers core body temperature and increases running performance. As shown herein, a composition comprising Lactobacillus reuteri MM2-3, Lactobacillus plantarum WCSF1, Streptococcus thermophilus B of R and Escherichia coli Nissle 1917 increased running time by about 1.5-fold over untreated. The composition also reduced core temperature during exercise by about 1° C.

In some embodiments, the composition reduces intestinal epithelial layer permeability. As shown herein, a comprising Lactobacillus reuteri MM2-3, Lactobacillus plantarum WCSF1, Streptococcus thermophilus B of R and Escherichia coli Nissle 1917 reduces epithelial layer permeability under inflammation in an inflammatory bowel disease model compared to untreated.

In some embodiments, the subject is human.

In a third aspect, the present invention provides a composition defined in the first aspect for use in a method of treating or ameliorating conditions associated with intestinal barrier dysfunction and/or heat stress in a subject.

In a fourth aspect, the present invention provides a use of a composition defined in the first aspect for the manufacture of a medicament for the treatment or prophylaxis of intestinal barrier dysfunction and/or heat stress in a subject.

In some embodiments, the treatment is for heat stress-induced gut permeability.

In some embodiments, the medicament lowers core body temperature and increases running performance of the subject.

In some embodiments, the medicament is for a human.

Having now generally described the invention, the same will be more readily understood through reference to the following examples which are provided by way of illustration, and are not intended to be limiting of the present invention.

### BRIEF DESCRIPTION OF THE FIGURES

FIG. 1 shows the analysis on expression of tight junction protein ZO-1 in Caco-2 cells co-cultured with single probiotics strains (A) and probiotics cocktail (B) against heat stress. Western blot was performed for analyzing ZO-1 expression (fold change) normalized to beta-actin protein, a housekeeping protein. The ZO-1 expression was set to 1. The 4-strain cocktail at a 10⁷ CFU/mL, which comprises of L. reuteri MM2-3, L. plantarum WCSF1, S. thermophilus and E. coli Nissle 1917 Eda at equal CFU/mL), was used for the assays.

FIG. 2 shows core body temperature (A) and length of run time (B) in an animal model. Individual animals in each group were indicated with an “X”, with the control group on the left and the treatment group on the right. Animals treated with the probiotics cocktail (right) exhibited lower average core temperature and exercised for a longer period of time, compared to untreated animals (blue) (p<0.05).

FIG. 3 shows the measurement of FITC-dextran concentration in blood serum of animals given with the probiotics cocktail (orange) and the untreated animals (blue) that were subjected to running exercise. Individual animals in each group were indicated with an “X”. Significant increase in the concentration of FITC-dextran was observed in the serum of untreated animals post exercise, compared to animals given the probiotics cocktail, indicating an increased gut permeability in the negative control animals (p<0.01).

FIG. 4 shows the immunofluorescent staining of colorectal tissue from the PBS-treated animals and animals treated with the probiotics cocktail after subjected to running exercise. Nucleus (blue) was stained by DAPI, while tight junction proteins occludin (indicated by an arrow) and ZO-1 (red) were detected by their respective anti-bodies conjugated with fluorescent probes. The fluorescence was observed under a fluorescence microscope.

FIG. 5 shows the histological assessment of inflammation in colon tissues by H&E staining. Colon tissues were collected on day 11, strained and observed under microscopy. Control, without DSS.

FIG. 6 shows a graph of the permeability of an inflamed Caco-2 epithelial layer co-cultured with Naïve CD4⁺ T cells. The concentration of FD10 that passed through from the apical to the basal compartment was measured after treatment with either the probiotic of the invention or a commercial probiotic. Significance (P<0.05, T-test) was indicated by a star. Error bar represents standard deviation of two independent experiments.

### DEFINITIONS

Certain terms employed in the specification, examples and appended claims are collected here for convenience.

As used in the specification and the appended claims, the singular forms “a,” “an” and “the” include plural referents unless the context clearly dictates otherwise.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

A bacterial “strain” as used herein refers to a bacterium which remains genetically unchanged when grown or multiplied.

Bibliographic references mentioned in the present specification are for convenience listed in the form of a list of references and added at the end of the examples. The whole content of such bibliographic references is herein incorporated by reference but their mention in the specification does not imply that they form part of the common general knowledge.

### DETAILED DESCRIPTION OF THE INVENTION

### Examples

Standard molecular biology techniques known in the art and not specifically described were generally followed as described in Green and Sambrook, Molecular Cloning: A Laboratory Manual, Cold Springs Harbor Laboratory, New York (2012).

### Example 1

### Materials and Methods

### Probiotic Strains

A panel of probiotic strains (Table 1) was cultured for approximately 16 hours before co-cultured with human colorectal cells (Caco-2) or before administered orally in mice. Probiotics cultures were briefly washed in sterile 1×PBS and centrifuged (4000 rpm, 4 minutes) thrice before OD600 reading were taken using a spectrophotometer. The colony forming unit (CFU) of each strain was then calculated and cells were diluted to the desired CFU in sterile 1×PBS. For in vitro co-culture studies, 10⁴, 10⁵, and 10⁶ CFU/mL per strain were used. For oral administration in mice, L. reuteri MM2-3, L. plantarum WCSF1, S. thermophilus and E. coli Nissle 1917 at 5×10⁸ CFU/mL per strain were mixed to attain the probiotics cocktail.

TABLE 1
List of probiotics strains used for in vitro
screening and cultivation conditions

| | | | Incubation |
|---|---|---|---|
| Strain | Medium | Temperature | mode |
| Escherichia coli Nissle | Lauria-Bertani | 37° C. | Shaking |
| 1917 Eda¹¹ | (LB) Broth | | |
| Lactobacillus rhamnosus | Lactobacillus MRS | 37° C. | Stationary |
| GG (ATCC 53103) | Broth (BD288130) | | |
| Saccharomyces boulardii | Difco YPD Broth | 30° C. | Shaking |
| (ATCC 74012) | | | |
| Lactobacillus reuteri | Lactobacillus MRS | 37° C. | Stationary |
| (DSM 20016) | Broth (BD288130) | | |
| Lactobacillus reuteri | Lactobacillus MRS | 37° C. | Stationary |
| MM 2-3 (PTA-4659) | Broth (BD288130) | | |
| Lactobacillus plantarum | Lactobacillus MRS | 37° C. | Stationary |
| (ATCC 14917) | Broth (BD288130) | | |
| Lactobacillus plantarum | Lactobacillus MRS | 37° C. | Stationary |
| WCSF1 (ATCC BAA- | Broth (BD288130) | | |
| 793) | | | |
| Streptococcus | M17 Broth (Sigma | 37° C. | Shaking |
| thermophilus B of R | 56156) | | |
| (DSM 20617) | | | |

### Co-Culturing of Caco-2 and Probiotics Strains

Caco-2 was maintained in DMEM (Life Technologies) supplemented with 15% FBS and 0.1% v/v antibiotics (penicillin/streptomycin) in a humidified environment (37° C., 5% CO₂, 95% air). Co-cultures with probiotics strain were carried out in 15% FBS (BioWest) supplemented with DMEM without antibiotics and cultured a humidified environment (41° C., 5% CO₂, 95% air) for 24 hours before Caco-2 cells were harvested for protein extraction.

### Western Blotting Analysis

After 24 hours co-culture of Caco-2 cells and the respective probiotic strains, cell culture supernatant was removed and Caco-2 cells were gently washed with 1×PBS. One hundred microlitres of ice-cold 1% Triton-X cell lysis buffer (150 mM NaCl, 1% Triton-X, 50 mM Tris pH 8.0) was added to each sample and cells were lysed on ice. The lysate was centrifuged for 15 minutes at 12000 rpm at 4° C. The supernatant was collected and boiled for analysis on a 10% SDS-PAGE gel. Anti-zona occludens 1 (anti-ZO1) antibody (Life Technologies) were used to detect the protein bands that were transferred from the SDS-page gel to 0.4 μm PVDF membrane.

### Pre-Clinical Animal Experiment

BALB/c mice (male, 8-9 weeks, 21-25 grams, In Vivos) were acclimatized for at least three days followed by subcutaneous implantation (SC) of real-time telemetry temperature loggers “Anipill” (Data Sciences International). For comparison of actual core temperature, five animals were subjected to intraperitoneal implantation (IP) of the loggers. The difference in the average of SC and IP temperatures logged was added to all subsequent SC temperature readings obtained in the animals. The animals were allowed to recover for at least 14 days post-surgery. They were then subjected to a treadmill training exercise, adapted from a previously published mouse treadmill fatigue test [Dougherty, J. P. et al. Journal of Visualized Experiments: JoVE, doi:10.3791/54052 (2016)], for five consecutive days. The animals were then allowed to rest for another three days before the actual treadmill running experiment conducted in an environmental chamber set at 32° C. with relative humidity (RH) at 60-80%.

Approximately 18 hours before the running experiment, a group of animals was orally gavaged with sterile PBS, while another group was given with the probiotics cocktail at 10⁹ CFU/animal. The animals then fasted for 3 hours before the running experiment and orally gavaged with 4 kDa FITC-dextran (Sigma-Aldrich) an hour before the running experiment. For the running experiment, the animals were removed from the treadmill either when the logged body temperature reached 41° C., or when the animals rest at the end of the treadmill for at least five seconds, or when the animal ran for 40 minutes, whichever happened first. All animals were sacrificed immediately after the running experiment. Trunk blood and large intestines were removed for subsequent blood serum and histological staining assays.

We treated mice with 2% Dextran Sulfate Sodium (DSS) on day 8-15. Colon tissues were collected for histological assessment of inflammation by hematoxylin and eosin (H&E) staining. All procedures involving animals were approved by IACUC (ref no. R19-0435 and 2020/SHS/1614).

### Blood Serum Analysis

Ice-cold anti-coagulant (30% v/v) was added to all blood samples immediately after removal from each animal. The blood samples were mixed gently by inverting the tubes for a few times before centrifuging for 30 minutes at 2000 rpm and 4° C. Serum was then carefully removed and 10 μl of serum was used for FITC-dextran analysis using a microplate reader at excitation 475 nm/emission 515 nm (BioTek).

### Histology and Confocal Microscopy

The large intestine of each animal was removed and washed with sterile 1×PBS several times until clean. Each sample was then carefully coiled and placed in ice-cold 4% paraformaldehyde (PFA) in sucrose and fixed at 4° C. overnight on a rotator. The samples were then washed in 30% sucrose the next day for at least 12 hours. Washed sections were carefully removed and embedded in optimal cutting temperature compound (OCT) before storage at −80° C., until ready for sectioning by cryostat. Frozen intestine samples were sectioned at a thickness of 7 μm and mounted on clean microscope slides. Excess OCT around the sections was allowed to thaw and evaporate at room temperature. Samples were blocked with 0.1% BSA for 30 minutes before incubation with anti-occludin (Life Technologies) and anti-ZO1 antibodies overnight at 4° C. Samples were then incubated for 1 hour at room temperature with fluorescence probe-conjugated secondary antibodies (Cell Signaling) the next day, and lastly stained with nuclear stain DAPI (NucBlue™ Fixed Cell ReadyProbes™ Reagent, LifeTech) for 3 minutes before clean coverslip was mounted over each sample. Samples were gently washed with 0.1% TBST in between each incubation as well as before and after nuclear staining. Prolong Gold anti-fade mountant (LifeTech) was used to preserve the fluorescence in each sample.

### Example 2

Expression of Tight Junction Protein in Caco-2 Increases when Co-Cultured with Probiotic Strains

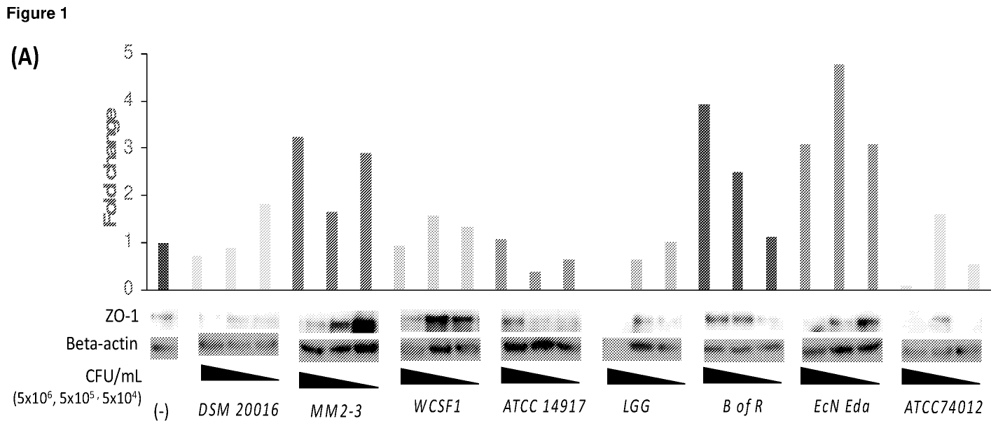

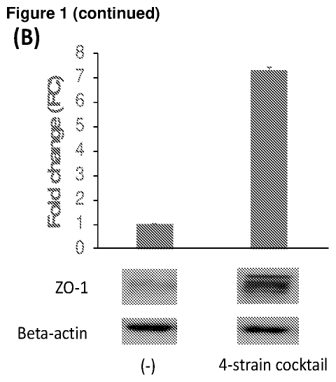

Caco-2 cells grown to a confluent state were co-cultured with the aforementioned probiotic strains for 24 hours at 41° C. to simulate a heat-stress condition. Western blot was carried out to detect the differential expression of tight junction protein ZO-1 in the cell culture with or without probiotics. ZO-1 is a scaffold protein involved in the formation of various tight junctions that hold epithelial cells together [Fanning, A. S. et al., J Biol Chem 273: 29745-29753 (1998)]. Several studies have shown that the expression of ZO-1 protein is temperature and time-dependent [Dokladny, K. et al., Am J Physiol Gastrointest Liver Physiol 290: g204-212 (2006); He, S. et al. PLoS One 11: e0145236 (2016)]. FIG. 1A shows that four probiotics strains L. reuteri MM2-3, L. plantarum WCSF1, S. thermophilus and E. coli Nissle 1917 Eda respectively increased ZO-1 expression by 1.3-4.8 folds at two or three different concentrations under a heat stress condition (41° C. for 24 hours). Further, FIG. 1B shows that a 4-strain cocktail that comprises all of L. reuteri MM2-3, L. plantarum WCSF1, S. thermophilus and E. coli Nissle 1917 Eda 4 increased ZO-1 expression by over 7 folds, which is 1.5-5.5 folds higher than the increases observed with the individual strains.

### Example 3

### Probiotics Cocktail Ameliorates Heat-Stress Induced Gut Permeability in an Animal Model

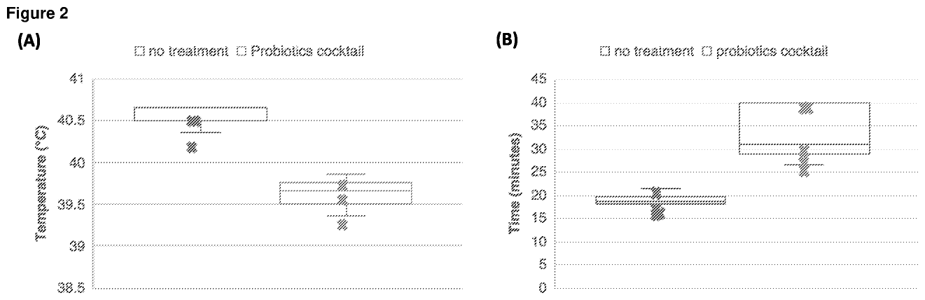

Next, we validated the protective effect of the 4-strain cocktail in an animal model. To this end, we mixed four probiotics strains L. reuteri MM2-3, L. plantarum WCSF1, S. thermophilus and E. coli Nissle 1917 at 5×10⁸ CFU/strain and administered the cocktail to mice subjected to running exercise in a hot and humid environment (32° C., RH 60-80%). The main markers for endurance used in this study are core body temperature and length of running time. FIG. 2 shows that animals treated with the probiotics cocktail had ˜1° C. lower core temperature and 1.5-fold longer run time. We also found that it took 6 min longer for the probiotics cocktail-treated mice to reach the peak core temperature than the PBS-treated controls. These results reveal that the treatment by our probiotic cocktail improved mice's physical performance over the PBS-treated controls against heat stress.

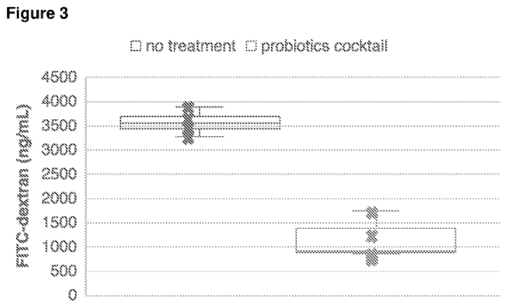

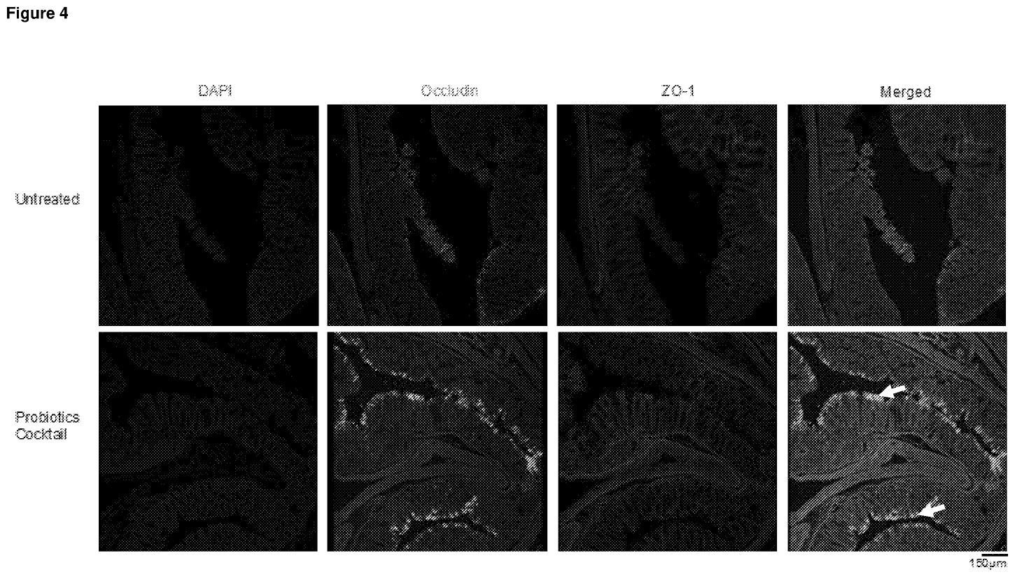

We also examined the effects of the 4-strain probiotics cocktail on gut epithelium permeability. “Leaky gut” syndrome occurs when the gut epithelial lining is compromised, allowing an increased efflux of molecules and toxins to enter the bloodstream from the gut. An increase in core body temperature may compromise gut epithelial integrity, causing the “leaky gut” syndrome [Yang, P. C., et al., J Gasl Hepatol 22: 1823-18311111(2007)]. We administrated a fluorescent-probed small molecule FITC-dextran one hour prior to exercise by oral gavage and measured its concentration in the blood serum collected after the running experiments. An increased concentration of FITC-dextran in the blood serum can be detected if the gut epithelium is compromised. FIG. 3 shows a 2-fold lower concentration of FITC-dextran in the blood serum of animals treated with the probiotics cocktail than that in the PBS-treated animals. This result indicates a reduction of gut permeability in the animals given the probiotic cocktail. Further, FIG. 4 shows increased expression of tight junction proteins occludin and ZO-1 in the gut mucosal epithelial in animals treated with the probiotics cocktail, suggesting improvement of the gut tight junction over the PBS-treated animals against heat stress.

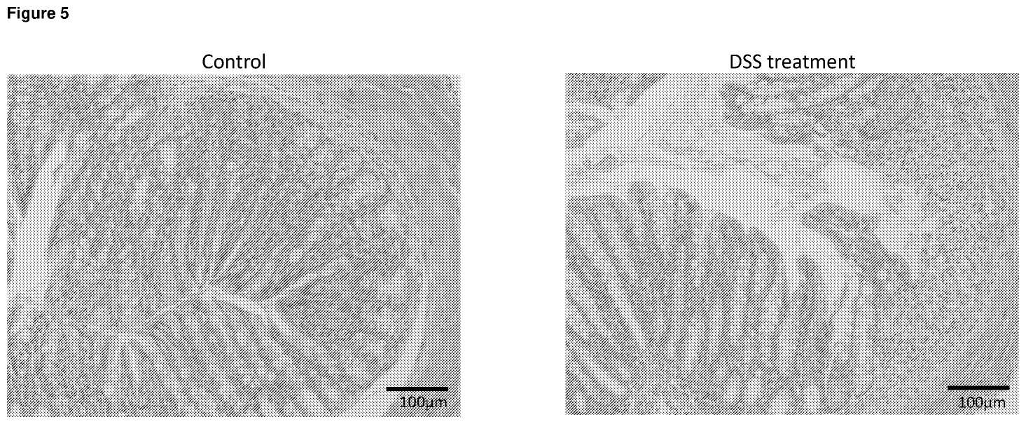

To allow further validation of our probiotics cocktail's functionality, we have established an in vivo model of Inflammatory Bowel Disease (IBD). We observed decreased intestine length, together with ulceration and crypt-cell loss in the colon tissues of DSS-treated mice, suggesting that inflammation was successfully induced by DSS treatment (FIG. 5).

### Example 4

### Probiotics Cocktail Reduced the Permeability of the Inflamed Epithelial Layer in an In Vitro Inflammatory Bowel Disease (IBD) Model

The ability of the probiotics composition of the invention to treat an intestinal barrier dysfunction disease such as IBD was tested in an in vitro inflammatory bowel disease (IBD) model.

Briefly, 5×10⁴/cm² Caco-2 cells were seeded on transwells and polarization was induced for approximately 2 weeks. Transepithelial electrical resistance (TEER) was measured after about 12 d of polarization and the polarized cells were ready for subsequent assays when the resistance was between 800-1000 Ohms/cm². Naïve CD4⁺ T cells were thawed and maintained in hlL-2 (600 U/mL) supplemented-ImmunoCult™ XF medium (StemCell Technologies) approximately 3 d before co-culturing with polarized Caco-2 cells. The Caco-2/T cells co-cultures were inflamed with an inflammation cocktail (interleukin 1-beta (rhIL-1β, 25 ng/ml), tumor necrosis factor alpha (rhTNF-α, 50 ng/ml), interferon gamma (rhIFN-γ, 50 ng/ml) and lipopolysaccharide (LPS, 1 μg/ml)) added to the apical compartment (Caco-2), while the T cells were cultured in the basal compartment. Inflammation was allowed for 36 h before respective treatments were supplemented to the inflamed transwells.

Treatments using 10⁷ CFU/mL of probiotics were carried out for 10 h. Supernatants from both apical and basal compartments were removed and 1 μg/mL of 10 kDa FITC-dextran (FD10) was diluted in fresh cell culture medium and applied to the apical compartment at the end of each indicated treatment. Sterile 1×PBS was supplemented to the basal compartment. The culture was incubated at 37° C. in a humidified 5% CO₂ atmosphere for 1 h before 100 μL of supernatant was removed from both apical and basal compartment. Fluorescence intensities were determined at an excitation wavelength of 485 nm and emission wavelength of 525 nm using a Synergy H1 microplate reader (BioTek).

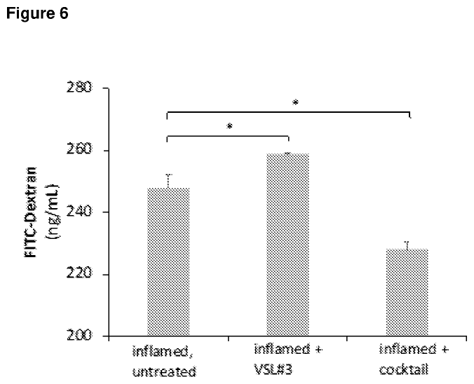

Results show that the treatment of our probiotics cocktail significantly resulted in 8.1% lower FD10 in the basal compartment, suggesting permeability reduction compared to control without treatment (FIG. 6). A comparative treatment, using a commercially available probiotic VSL #3 (also namely Vivomixx), led to 4.5% higher FD10 in the basal compartment. In summary, our results show that our probiotics cocktail exert a protective effect on the epithelial layer under inflammation in the in vitro IBD model, which is advantageous over VSL #3.

### SUMMARY

We verified the probiotic strains that increase the expression of tight junction protein ZO-1. Using the verified strains, we developed a novel 4-strain probiotic cocktail and confirmed the protective effects of the cocktail against intestinal barrier dysfunction and heat stress in an animal model. The 4-strain cocktail (L. reuteri MM2-3, L. plantarum WCSF1, S. thermophilus B of R and E. coli Nissle 1917) is novel and able to promote expression of tight junction proteins, reduce gut permeability and improve physical performance (lower core body temperature and longer running time) in a hot and humid environment. Our probiotics cocktail is applicable to addressing health issues that arise from intestinal barrier dysfunction and heat stress, and to promoting physical performance under heat stress.

### REFERENCES

Cheng, F. S., Pan, D., Chang, B., Jiang, M. & Sang, L. X. Probiotic mixture VSL #3: An overview of basic and clinical studies in chronic diseases. World J Clin Cases 8, 1361-1384, doi:10.12998/wjcc.v8.i8.1361 (2020).
Cinque, B. et al. Production Conditions Affect the In Vitro Anti-Tumoral Effects of a High Concentration Multi-Strain Probiotic Preparation. PLoS One 11, e0163216, doi:10.1371/journal.pone.0163216 (2016).
Clark, A. & Mach, N. Exercise-induced stress behavior, gut-microbiota-brain axis and diet: a systematic review for athletes. J Int Soc Sports Nutr 13, 43, doi:10.1186/s12970-016-0155-6 (2016).
Dokladny, K., Moseley, P. L. & Ma, T. Y. Physiologically relevant increase in temperature causes an increase in intestinal epithelial tight junction permeability. Am J Physiol Gastrointest Liver Physiol 290, G204-212, doi:10.1152/ajpgi.00401.2005 (2006).
Dougherty, J. P., Springer, D. A. & Gershengorn, M. C. The Treadmill Fatigue Test: A Simple, High-throughput Assay of Fatigue-like Behavior for the Mouse. Journal of Visualized Experiments: JoVE, doi:10.3791/54052 (2016).
Fanning, A. S., Jameson, B. J., Jesaitis, L. A. & Anderson, J. M. The tight junction protein ZO-1 establishes a link between the transmembrane protein occludin and the actin cytoskeleton. J Biol Chem 273, 29745-29753, doi:10.1074/jbc.273.45.29745 (1998).
He, S. et al. Protective Effects of Ferulic Acid against Heat Stress-Induced Intestinal Epithelial Barrier Dysfunction In Vitro and In Vivo. PLoS One 11, e0145236, doi:10.1371/journal.pone.0145236 (2016).
Kumar, M., Kissoon-Singh, V., Coria, A. L., Moreau, F. & Chadee, K. Probiotic mixture VSL #3 reduces colonic inflammation and improves intestinal barrier function in Muc2 mucin-deficient mice. Am J Physiol Gastrointest Liver Physiol 312, G34-G45, doi:10.1152/ajpgi.00298.2016 (2017).
Moran, A. P., Prendergast, M. M. & Appelmelk, B. J. Molecular mimicry of host structures by bacterial lipopolysaccharides and its contribution to disease. FEMS Immunol Med Microbiol 16, 105-115, doi:10.1111/j.1574-695X.1996.tb00127.x (1996).
Shing, C. M. et al. Effects of probiotics supplementation on gastrointestinal permeability, inflammation and exercise performance in the heat. Eur J Appl Physiol 114, 93-103, doi:10.1007/s00421-013-2748-y (2014).
Yang, P. C., He, S. H. & Zheng, P. Y. Investigation into the signal transduction pathway via which heat stress impairs intestinal epithelial barrier function. J Gastroenterol Hepatol 22, 1823-1831, doi:10.1111/j.1440-1746.2006.04710.x (2007).

1. A composition comprising or consisting of two or more of Lactobacillus reuteri MM2-3, Lactobacillus plantarum WCSF1, Streptococcus thermophilus B of R and Escherichia coli Nissle 1917, formulated to prevent or treat intestinal barrier dysfunction and/or heat stress in a subject.

2. The composition of claim 1, comprising or consisting of Lactobacillus reuteri MM2-3, Lactobacillus plantarum WCSF1, Streptococcus thermophilus B of R and Escherichia coli Nissle 1917.

3. The composition of claim 1, wherein the heat stress is at least in part due to physical exertion by the subject.

4. The composition of claim 1, formulated to prevent or treat heat stress-induced gut permeability in said subject.

5. The composition of claim 4, comprising a dose of about 10⁸ colony-forming units (CFU) of each strain.

6. The composition of claim 1, wherein the subject is human.

7. The composition of claim 1, wherein the composition is in the form of a gelatin capsule, pressed tablet, gel cap, liquid beverage or sachet.

8. A method of treatment or prophylaxis comprising administering to a subject in need of such treatment or prophylaxis an efficacious amount of a composition of claim 1.

9. The method of claim 8, wherein the subject has, or will get, intestinal barrier dysfunction and/or heat stress.

10. The method of claim 8, wherein the subject is administered a composition comprising at least 10⁸ to 10¹¹ CFU per day.

11. The method of claim 10, wherein the subject is administered a composition comprising at least 10⁸ CFU of each strain, preferably at least 5×10⁹ CFU of each strain.

12. The method of claim 8, wherein the composition ameliorates intestinal barrier dysfunction and/or lowers core body temperature and increases running performance.

13. The method of claim 8, wherein the subject is human.

14. A composition of 1, for use in a method of treating or ameliorating conditions associated with intestinal barrier dysfunction and/or heat stress in a subject.

15.-18. (canceled)

19. The composition of claim 2, for use in a method of treating or ameliorating conditions associated with intestinal barrier dysfunction and/or heat stress in a subject.
